(19) United States
(12) Patent Application Publication
(10) Pub. No.: US 2014/0067038 A1
(43) Pub. Date: Mar. 6, 2014

(54) DEVICES AND SYSTEMS FOR RETAINING A MEDICAL DEVICE AT A TREATMENT SITE

(71) Applicant: W. L. GORE & ASSOCIATES, INC., Newark, DE (US)

(72) Inventors: John R. Daugherty, Flagstaff, AZ (US); Joel M. Greene, Flagstaff, AZ (US)

(73) Assignee: W. L. GORE & ASSOCIATES, INC., Newark, DE (US)

(21) Appl. No.: 14/012,086

(22) Filed: Aug. 28, 2013

Related U.S. Application Data
(60) Provisional application No. 61/694,691, filed on Aug. 29, 2012.

(51) Int. Cl. A61F 2/06

(52) U.S. Cl. CPC A61F 2/064
USPC 623/1.14; 156/272.8; 156/250

(57) ABSTRACT

In accordance with various embodiments, an anchoring system for a medical device comprises one or more biased hooks. The one or more biased hooks may be formed by any suitable process. Moreover, the one or more biased hooks may be formed from a shape memory material. The anchoring system may be processed in any suitable way to provide a designed or predefined failure mode. This failure mode may be designed to protect or prevent damage to the medical device. The anchoring system may be configured with a plurality of hooks biased in various directions. Moreover, the anchoring system may be configured with a plurality of substantially small hooks configured to engage the anatomy at multiple points. As such, the anchoring systems may be customizable and provide for an implantable medical device with a reduced delivery geometry and/or deployment geometry.

### BACKGROUND

1. Field

This disclosure relates to devices and systems for retaining medical devices and more specifically, to anchoring devices and systems that are attachable to or integrally formed in a medical device, and that are capable of engaging the anatomy or other suitable structures.

2. Discussion of the Related Art

Typical medical devices that comprise retention mechanisms or anchoring devices generally have large pre-deployment package geometries making deployment cumbersome. These larger geometries require large, disruptive therapeutic procedures. Further, typical anchoring systems are not sufficiently customizable or configurable for various medical devices, and/or installations.

Thus, a need exists for customizable anchoring systems and for reducing the delivery and deployment geometry of implantable medical devices with anchoring systems.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the drawing figures, wherein like numerals denote like elements and wherein:

FIGS. 1A-1C illustrate top views of hooks in various configurations;

FIGS. 2A-2E illustrate perspective views of hooks in various configurations;

FIGS. 3A-3E illustrate top views of hooks comprising a plurality of tines in various configurations;

FIG. 3F illustrates a profile view of a medical device comprising a hook;

FIG. 3G illustrates a profile view of a medical device comprising a plurality of hooks;

FIG. 3H illustrates a perspective view of a hook comprising one or more tines biased in a direction;

FIGS. 4A-4B illustrate a perspective view of a hook;

FIG. 5A illustrates a profile view of a wire comprising a plurality of hooks;

FIGS. 5B-5C illustrate perspective views of a wire comprising a plurality of hooks;

FIG. 5D illustrates a profile view of a medical device comprising integrally formed hooks; and

FIG. 5E illustrates a profile view of a cross-section of a portion of a medical device comprising integrally formed hooks.

### DETAILED DESCRIPTION OF THE ILLUSTRATED EMBODIMENTS

The detailed description of various embodiments herein makes reference to the accompanying drawing figures, which show various embodiments and implementations thereof by way of illustration and best mode, and not of limitation. While these embodiments are described in sufficient detail to enable those skilled in the art to practice the embodiments, it should be understood that other embodiments may be realized and that mechanical and other changes may be made without departing from the spirit and scope of the present disclosure. Furthermore, any reference to singular includes plural embodiments, and any reference to more than one component may include a singular embodiment. Moreover, recitation of multiple embodiments having stated features is not intended to exclude other embodiments having additional features or other embodiments incorporating different combinations of the stated features.

As used herein, “medical device” may include, for example stents, grafts, and stent-grafts, (whether single, bifurcated, etc.), valves, and drug-delivering devices, to name just a few, that are implanted, acutely or chronically, in the anatomy at a treatment region.

As used herein, “tine” is an engagement member that is configured to engage the anatomy or any other suitable structure and includes, for example, a point, prong, barb, serration, point, tooth, and/or the like.

The present disclosure relates to a number of non-limiting embodiments, each of which may be used alone or in coordination with one another. In various embodiments, an anchoring system may be any suitable system for retaining a medical device in a treatment region of the anatomy or to any suitable structure. For example, the anchoring system may include one or more hooks that are adhered or retained on a medical device. These hooks may comprise a body portion, a tine portion, and a hinge portion coupling the tine portion to the body portion. The hooks may have shape memory characteristics that allow the hooks to have a delivery geometry during transport to a treatment site and a deployment geometry that engages the anatomy when the medical device is implanted. The anchoring systems described herein may provide benefits, such as adaptability allowing hooks of various configurations to be installed in any suitable location and/or orientation on a medical device, improved delivery and deployment by providing a smaller delivery geometry, and customizability providing hooks of various configurations, geometries, sizes and the like.

In various embodiments, an anchoring system or one or more hooks may be configured to couple to or be integrally formed in a medical device. For example, an anchoring system may be adhered or fastened to a medical device. The anchoring system may also be integrally formed in the medical device such as, for example, hooks etched or otherwise formed in the interior or exterior surface of a stent. The anchoring system may be configured to engage the anatomy (e.g., the vasculature). The anchoring system may also be configured to engage a medical device or a cover (e.g., a graft material). As such, the various anchoring system described herein are (1) installable on any suitable medical device, (2) capable of engaging any suitable structure or surface, and (3) are customizable.

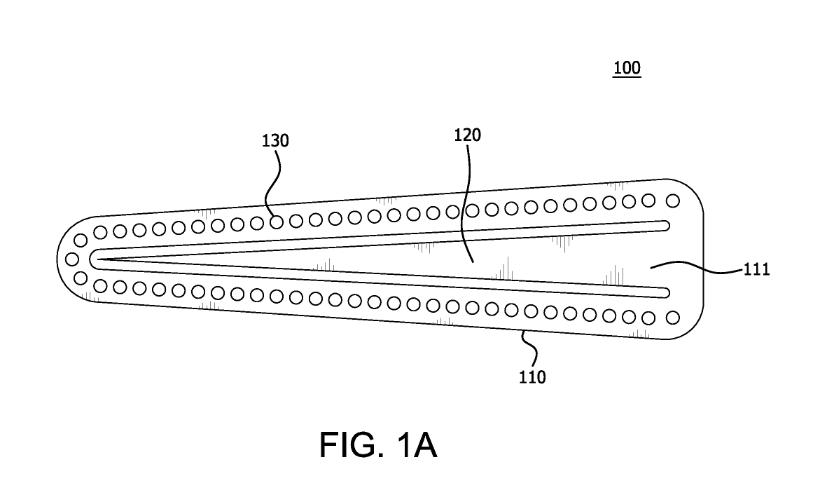

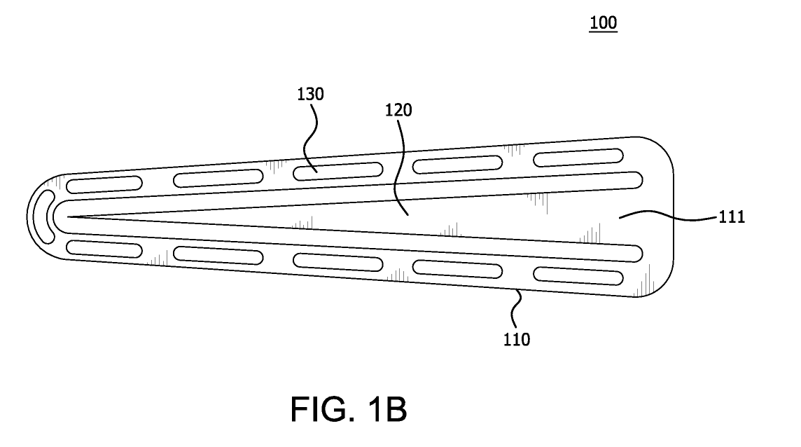

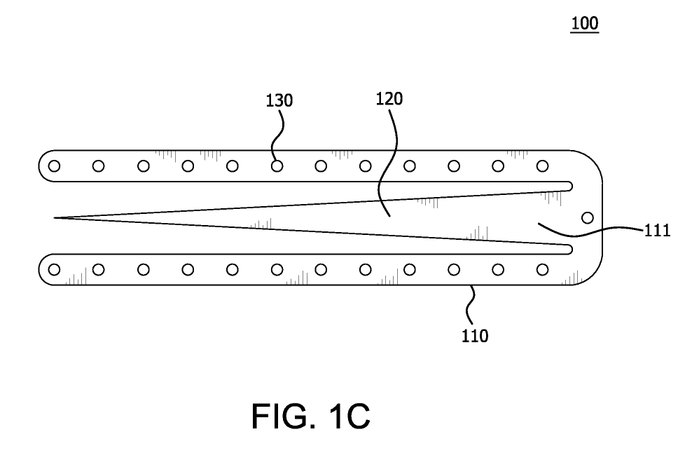

In various embodiments and with reference to FIGS. 1A-1C, an anchoring system may comprise one or more hooks 100. Hook 100 may be any suitable structure configured to couple and/or retain a medical device to an anatomy or other suitable structure. Moreover, hook 100 may be integrally formed on, attachable to, or installable on a medical device. Hook 100 may be any suitable size and/or shape. Hook 100 may comprise a body 110 and a tine 120. Body 110 and tine 120 may be joined or coupled to one another at hinge 111.

Tine 120 may be bent, biased, or otherwise shaped such that it protrudes from body 110 about hinge 111. Stated another way, a first plane defined by a top surface of tine 120 intersects a second plane defined by the top surface of body 110. The angle defined by the intersecting planes may be any suitable angle, such that tine 120 engages and is retained in the anatomy or other suitable structure when a medical device is implanted at a treatment region.

Tine 120 may be flexibly mounted to body 110. Moreover, tine 120 may have shape memory properties. Prior to implanting the medical device in the anatomy, tine 120 may be depressed or bent such that the first plane defined by the top surface of tine 120 is in substantially the same plane as the second plane defined by the top surface to the body 110. Tine 120 may be retained or stay in this substantially planar configuration during delivery of a medical device to a treatment region in the anatomy. For example, the medical device and attached hook 100 may be covered by a sleeve or other suitable cover configured to restrain the medical device in a delivery configuration as it passes through the anatomy to a treatment region. Prior to deployment, the sleeve or cover may be removed allowing the medical device and/or tine 120 to expand to a deployment configuration.

Tine 120 may also have shape memory properties that allow tine 120 to be deformed (e.g., compressed) for delivery and then expanded by a triggering mechanism. The triggering mechanism may be heat including for example, the heat from the body conducted to tine 120 at the treatment region by the anatomy. The triggering mechanism may also be any other suitable trigger including, for example, removing a cover, an electric current, ultrasonic energy, a warm solution flush, and/or the like.

Body 110 may further comprise one or more holes 130. Hole 130 may be of any suitable size and/or shape. Hole 130 in body 110 may be provided to facilitate adhering hook 100 to a medical device. For example, where hook 100 is coupled to a medical device with an adhesive, the hole 130 allows the adhesive to create a stronger bond between hook 100 and the medical device. More specifically, the adhesive may adhere to the interior surfaces of hole 130 to better resist stresses created on hook 100 when implanted in the anatomy.

Body 110 may be of any suitable shape or size. For example, body 110 may have a closed profile, as shown in FIGS. 1A and 1B. In this configuration, tine 120 is completely enclosed by the profile of body 110. Body 110 may have an open profile, as shown in FIG. 1C. In this configuration, the point of tine 120 may not be enclosed by body 110. The open profile of body 110 may provide more clearance for tine 120 than with a closed profile.

In various embodiments, hook 100 may comprise body 110 with an open profile that provides additional clearance between tine 120 and the portions of body 110 adjacent to tine 120. Moreover, hook 100 may be mounted or coupled to a medical device comprising a coating or graft material including, for example, a fluoropolymer such as ePTFE. The additional clearance between tine 120 and the portions of body 110 adjacent to tine 120 may limit the amount of scissoring damage (e.g., movement between tine 120 and body 110) or wear to the coating or graft material that can be created during delivery of the medical device to the treatment region in the compressed delivery configuration.

As noted above, hinge 111 couples body 110 to tine 120. Moreover, hinge 111 is a portion of hook 100 that may flexibly, plastically, or resiliently deform. As noted above with respect to tine 120, hinge 111 may have shape memory material properties. In other words, under certain conditions (e.g., temperatures), hinge 111 may conform to a rigid orientation and under other conditions, hinge 111 may be relatively flexible and/or malleable (e.g., adaptable or formable). Under operating conditions where hinge 111 is set (e.g., has a rigid orientation), hinge 111 generally creates the out of phase orientation between the first plane defined by the top surface of tine 120 and the second plane defined by the top surface of body 110. As such, hinge 111 provides for the delivery geometry and deployment geometry discussed above.

In various embodiments, one or more hooks 100 may be of any suitable size. For example, hook 100 has a length, width or thickness of less than about 25 μm. One or more hooks 100 may be coupled to a medical device, such that the area of the medical device where the hooks are installed has a “hook density” (e.g., a number of hooks 100 per square mm). For example, hook 100 having a maximum length or width of about 10 μm with a hook to hook spacing of about 5 μm would have a hook density of about 4,356 hooks per square mm on a surface of the medical device. As such, medical devices can comprise various numbers of hooks 100 and associated hook densities, including for example, hook densities of about 100, about 500, about 1,000, about 1,600, about 2,000 about 3,000 about 4,000 about 5,000 about, about 7,000, or about 10,000 or more hooks per square mm.

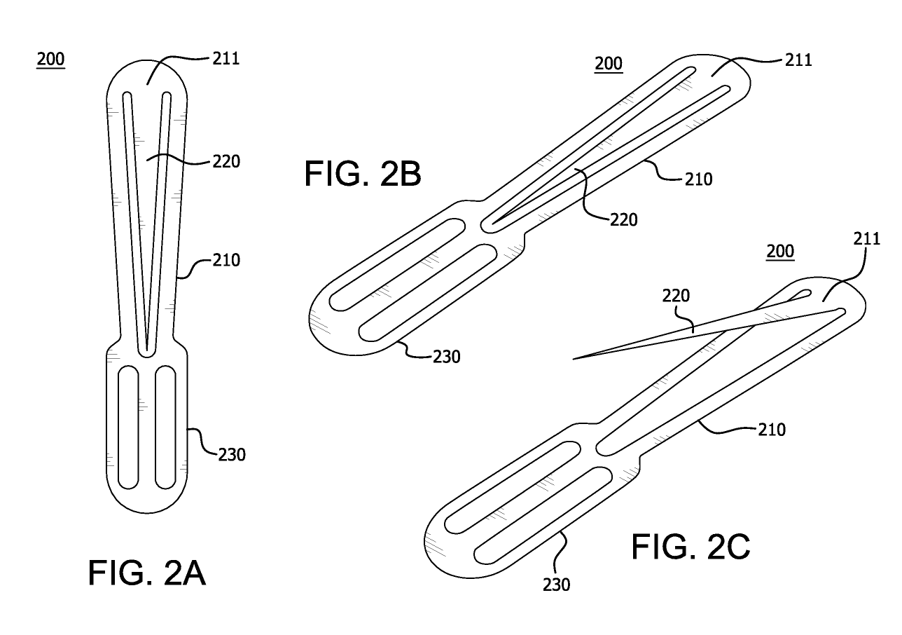

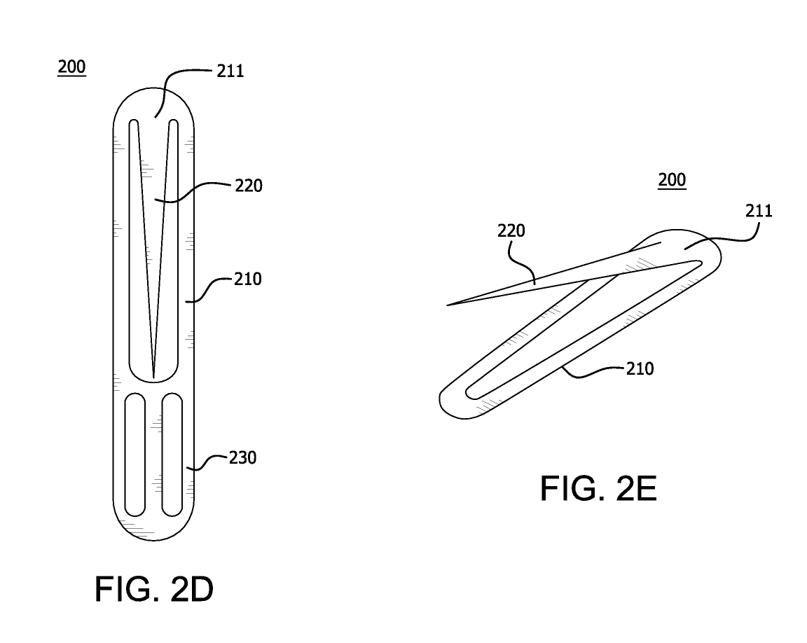

In various embodiments and with reference to FIGS. 2A-2D, hook 200 comprises a bracket or buckle 230. Bracket 230 may be a portion of body 210. Bracket 230 may be attached to or integrally formed in body 210. Hook 200 may be adhered (e.g., glued) and/or operatively coupled with an attachment material (e.g., wrapped with a graft material, implantable tape, and/or the like) to a medical device.

In various embodiments, bracket 230 may be configured to operatively receive a graft or adhering material. Bracket 230 may define one or more channels or holes separated by a retention member. The graft or adhering material (e.g., a fluoropolymer such as ePTFE) may thread or pass through the one or more channels and operatively engage the retention member. In this way, the graft material can be passed through the bracket and wrapped around the medical device such that the graft material used to retain the medical device is merged with graft material on a medical device such as a stent-graft. The graft material may, for example, pass through a first channel in bracket 230 in a first direction, engage a retention member, and pass through a second channel in bracket 230 in a second direction. The graft material may be pulled tight or integrated into a graft covering the medical device such that hook 200 is positively and/or integrally coupled to the medical device.

In various embodiments and as noted above, the area defined by body 210 adjacent tine 220 may vary by application. For example, in applications that are susceptible to scissoring or wear damage during delivery, the area may be enlarged to avoid wear or scissoring between an interior surface of body 210 and an exterior surface of tine 220, where tine 220 moves about hinge 211 relative to body 210. In applications where wear or scissoring is not an issue, the area defined by body 210 may be reduced to minimize the overall package of hook 200 or to increase the strength of body 210 by providing additional material at the transition between the portion of hook 200 defined by body 210 and bracket 230.

In various embodiments and with reference to FIG. 2E, hook 200 may comprise body 210 with a solid structure (e.g., no holes). This configuration may allow the overall package geometry of hook 200 to be smaller for certain applications where delivery and/or deployment geometry needs to be minimized.

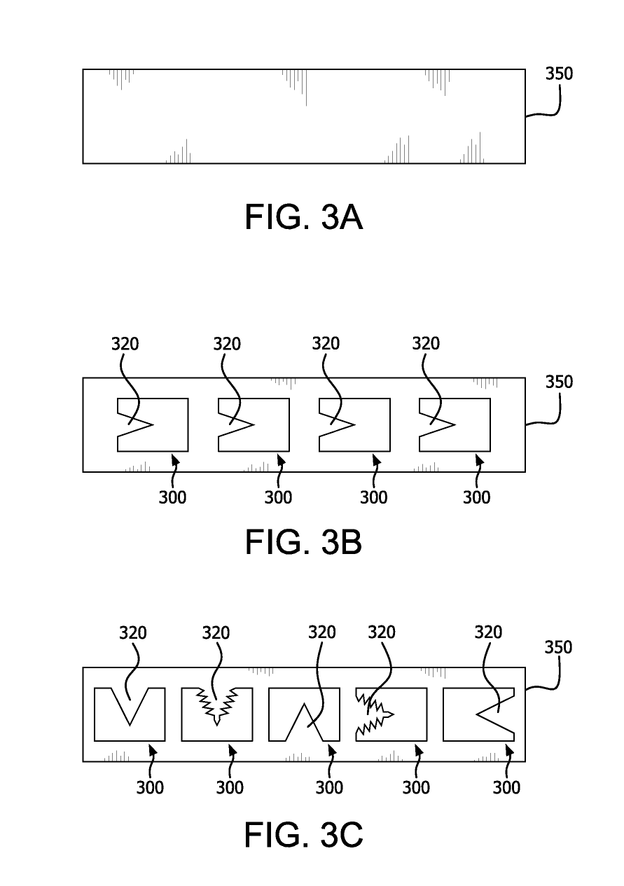

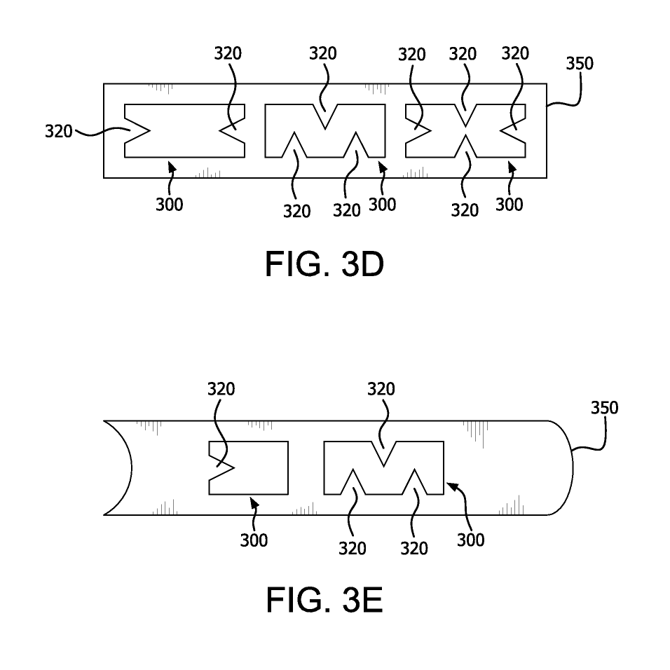

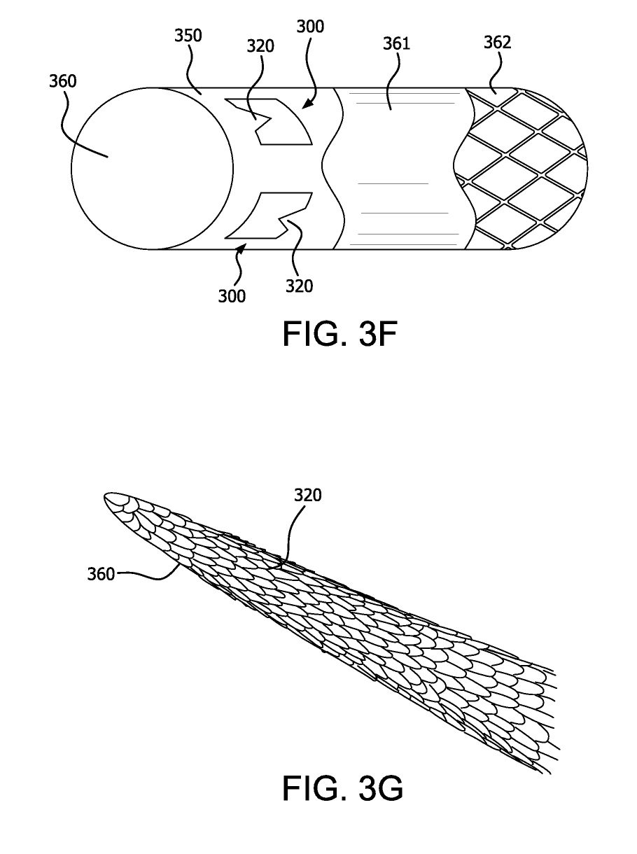

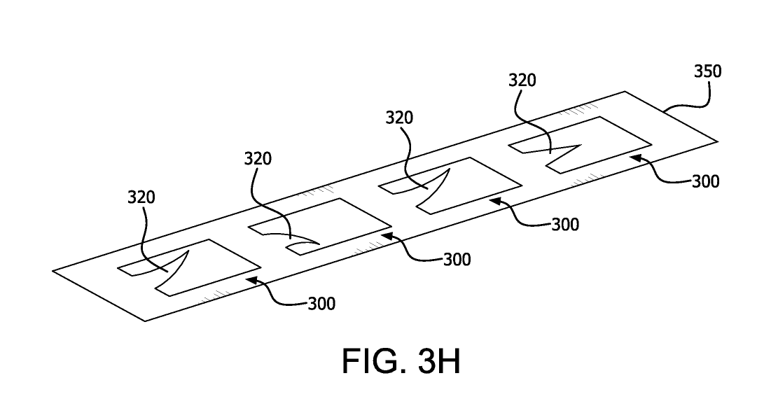

In various embodiments and with reference to FIGS. 3A-3E, anchoring system 350 may comprise one or more hooks 300 of any suitable shape, position, or orientation. One or more hooks 300 may comprise one or more tines 320. Tines 320 may be orientated in any suitable fashion. For example, tines 320 may have a uniform configuration, such that all tines 320 are oriented in substantially the same direction. Tines 320 may also be oriented in non-uniform directions. In these configurations, a plurality of tines 320 may be configured in opposing, or out of phase directions. For example, anchoring system 350 may comprise first tine 320 in a first direction and second tine 320 in a second opposing direction. First tine 320 and second tine 320 may be oriented such that they are substantially in phase with the longitudinal axis of anchoring system 350 or are out of phase with the longitudinal axis of anchoring system 350.

In various embodiments, tine 320 may be any suitable shape. For example, tine 320 may have a generally smooth profile. Tine 320 may also have a serrated, jagged, or non-smooth profile. The profile of tine 320 may be customizable, such that particular profile are selected or created based on the treatment region and composition of the anatomy or structure to be engage by tine 320. Moreover, the profile of tine 320 may be selected based on the properties of the medical device (e.g., weight, size, cross-sectional area, graft material, and/or the like) or properties of the anatomy (e.g., pressure, fluid flow rate, anatomy composition, and/or the like) where the medical device and associated one or more hooks 300 are installed.

In various embodiments and as noted above, anchoring system 350 may comprise one or more hooks 300. Similarly, hook 300 may comprise one or more tines 320. Hook 300 may comprise one or more tines 320 of any suitable shape and/or size in any suitable configuration and/or orientation. For example, hook 300 may comprise first tine 320 oriented in a first direction and having a first size and a first shape, second tine 320 oriented in a second direction and having a second size and a second shape, a third tine 320 oriented in a third direction and having a third size and a third shape, and the like. In another example, hook 300 may comprise a first tine 320, a second tine 320 and a third tine 320, each having the same orientation, shape and size. Moreover, hook 300 may comprise any number of tines 320 have any suitable orientation, shape, and size.

In various embodiments, anchoring system 350 and/or one or more hooks 300 may be provided as a system of multiple engagement elements or may be provided as individual engagement elements. These engagement elements may be attached to a medical device as single piece or as multiple pieces. Upon adherence or attachment to a medical device, anchoring system 350 or one or more hooks 300 may be integrally formed or operatively coupled to the medical device.

In various embodiments, anchoring system 350 and/or one or more hooks 300 may be formed in or cut from a sheet or tube of thin film material. For example, anchoring system 350 and/or one or more hooks 300 may be formed in a thin-film nitinol (hereinafter, “TFN”) or any other suitable material. A suitable material may comprise shape memory properties that allow the material to be formed or set in a first configuration (e.g., the deployment configuration) and to be deformed to a second configuration (e.g., the delivery configuration), such that the material returns to the first configuration in response to a triggering input (e.g., removal of a restraining element, heat, time, energy, and/or the like). The TFN may be formed in any suitable fashion. For example, anchors, hooks and/or tines may be cut from the TFN and then heat-treated. The anchors, hooks and/or tines may be shaped or formed and subjected to a second heat-treatment or may be shaped or formed prior to the initial heat-treatment. Exemplary, methods of forming and heat treating nitinol materials are disclosed in U.S. Pat. No. 7,811,393, entitled “Shape Memory Alloy Articles with Improved Fatigue Performance and Methods Therefor,” which is herein incorporated by reference in its entirety. For more information regarding the use of TFN in medical applications, see Rigberg, D., et al., JOURNAL OF VASCULAR SURGERY, Thin-film nitinol (NiTi): A Feasibility Study for a Novel Aortic Stent Graft Material (August 2009), which is herein incorporated by reference in its entirety.

In various embodiments and with reference to FIG. 3E, given the material properties of TFN or any other suitable material, anchoring system 350 and/or one or more hooks 300 may be configured or molded into any suitable shape. Hook 300 and/or anchoring system may be initially cut of shaped from a sheet or tube using any suitable method. For example, hook 300 and, more specifically, tine 320 may be cut or created in the TFN using a laser, photo-etching, a cutting tool, and/or other similar process.

The shape of anchoring system 350 and/or one or more hooks 300 may be selected based on the medical device that anchoring system 350 and/or one or more hooks 300 are adhered or coupled to for implantation. For example and in the context of a stent or stent graft, anchoring system 350 and/or one or more hooks 300 may be molded into a substantially or partially substantially cylindrical shape and/or may be cut from a tube having a generally cylindrical shape. In this way, one or more tines 320 may be formed such that tines 320 conform to the substantially cylindrical shape of anchoring system 350.

In various embodiments and with reference to FIG. 3F, a medical device 360 comprises a stent 362 and a graft 361. Medical device 360 may be coupled to or enclosed by anchoring system 350. For example, anchoring system 350 may be configured as a sleeve or cover. Anchoring system 350 may approximate or generally conform to the outer profile of medical device 360. Anchoring system 350 may further couple or mount to medical device 360 in any suitable fashion. As noted above, anchoring system 350 may be compressible to provide a delivery geometry that is suitable for insertion in the anatomy for placement at a treatment site. Anchoring system 350 may be expanded to a deployment geometry at the treatment site. Anchoring system 350 may be self-expanding or may expand in response to any suitable triggering condition. Once expanded, anchoring system 350 may be manipulated so that one or more tines 320 effectively retain medical device 360 in the anatomy so that medical device 360 does not move relative to the treatment site.

In various embodiments and with reference to FIG. 3G, medical device 360 may be covered by a plurality of tines 320. For example, medical device 360 may be an occluder or other suitable device with one closed end. The exterior surface of medical device 360 may be an anchoring system that is configured as a sleeve as discussed above. The sleeve may be comprised of a plurality of tines 320 that appear in a pinecone or Christmas-tree configuration. In this configuration, the plurality of tines 320 engage the anatomy at a plurality of points at the treatment region.

In various embodiments and with reference to FIG. 3H, anchoring system one of more tines 320 may be bent, formed, or biased in any suitable direction. Tine 320 may be biased such that the body portion of tine 320 has a substantially straight profile or is arced. Tine 320 may be shaped in any suitable fashion. For example, tine 320 may be bent on a mold or biased with a suitable shaping tool. Tine 320 may also be shaped or biased using laser shock peening where a high-energy laser is used to shape tine 320. Additional information regarding laser shock peening may be found in U.S. Patent Application Publication No. 2005/0182478, entitled Laser Shock Peening of Medical Devices, to Holman, et al. and U.S. Patent Application Publication No. 2009/0043228, entitled Laser Shock Peening of Medical Devices, to Northrop, et al., both of which are herein incorporated by reference in their entirety.

In various embodiments, hook 400 may be formed or shaped from TFN or another suitable material by any suitable process. For example, hook 400 and/or tine 420 may be formed in or from a sheet or tube of TFN by photo-etching. This process may include masking a geometry to define tine 420, hinge 411 and/or body 410 on a sheet of TFN. The sheet of TFN is then etched with a suitable solvent to remove the unmasked material. Hook 400 may then be initially heat treated or otherwise processed. Tine 420 and/or any other portion of hook 400 may be subjected to secondary processing. For example, tine 420 may be biased or otherwise bent out of phase using any suitable process, such as, laser peening as discussed above. An alternate process used to bias or bend an array of hooks out of phase, utilizes a high pressure fluid flow that is forced through the open portions of the hook array. The high pressure fluid flow can be used to deform (plastically, flexibly, or resiliently deform) the hook portions so they remain out of phase in a deployment and/or delivery configuration. Once tine 420 has been biased, hook 400 may be heat-treated or otherwise processed to achieve desired mechanical properties. Upon completion of processing, one or more hooks 400 may be adhered or otherwise coupled to a medical device in any suitable arrangement so that the medical device may be implanted and retained in the anatomy.

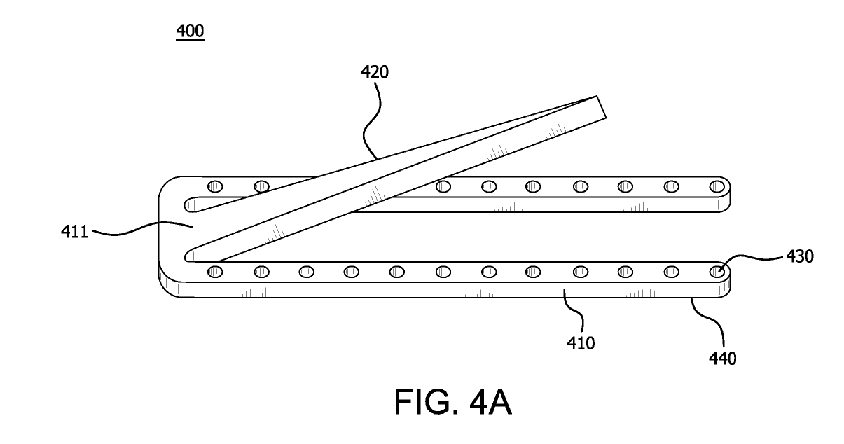

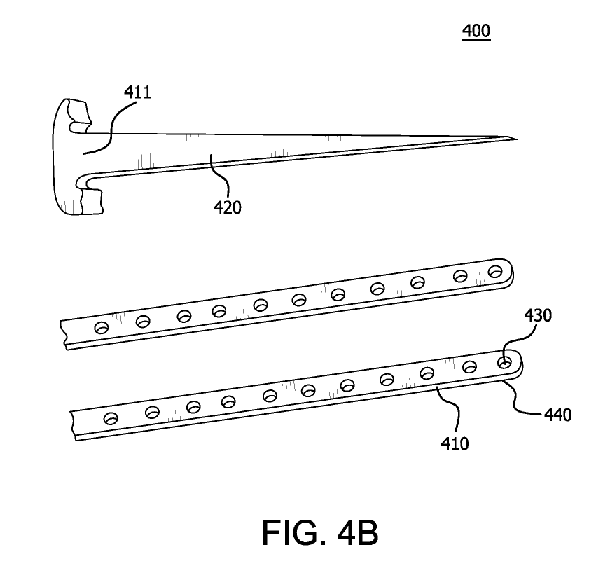

In various embodiments and with reference to FIGS. 4A and 4B, hook 400 may be configured with a geometry or a pre-stressed region to ensure a predefined failure mode in the event of an over-stress condition. More specifically, shear stress is exerted on a medical device and an associated anchor system when the medical device is implanted in the body. To insure the medical device is not damaged in an over-stress condition, hook 300 may be designed with a predetermined failure point or known failure mode. As noted above, tine 420 and body 410 are coupled together at hinge 411, creating a stress concentration based on the geometry. Tine 420 is biased out of phase with respect to body 410 at the region associated with hinge 411, increasing the overall stress concentration at hinge 411. The increase in stress concentration at hinge 411 creates a predefined or designed failure region to insure that the medical device is protected once it is implanted. In response to an over-stress condition, tine 420 may separate from body 410 in the region associated with hinge 411. Body 410 stays attached to the medical device and tine 420 is lodged in the anatomy associated with the treatment region. As such, damage or tearing of the medical device is limited or avoided completely.

In various embodiments, hook 400 may be capable of being coupled to any suitable medical device by any suitable method. For example, hook 400 may be adhered to a suitable medical using a film, adhesive, glue or other bio-compatible coating including, for example, FEP, and/or other commonly known bio-compatible thermo-plastics. Body 410 may comprise a bottom portion or bottom surface 440. Hook 400 may be coupled to or adhered to a medical device along bottom surface 440 and into holes 430 or along any other suitable surface.

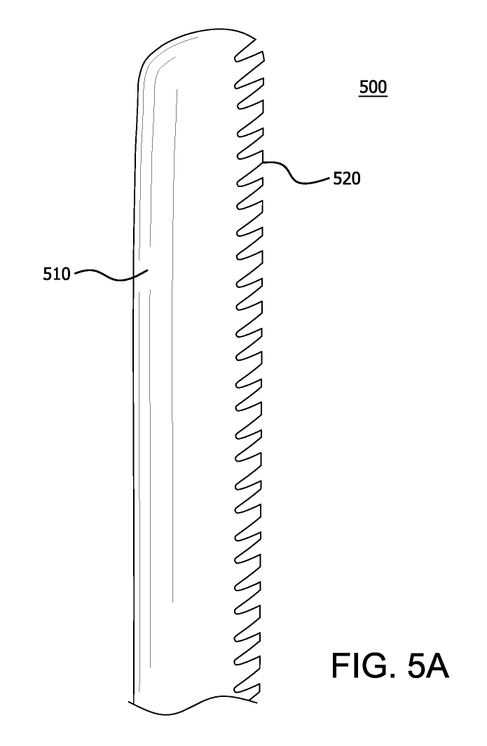

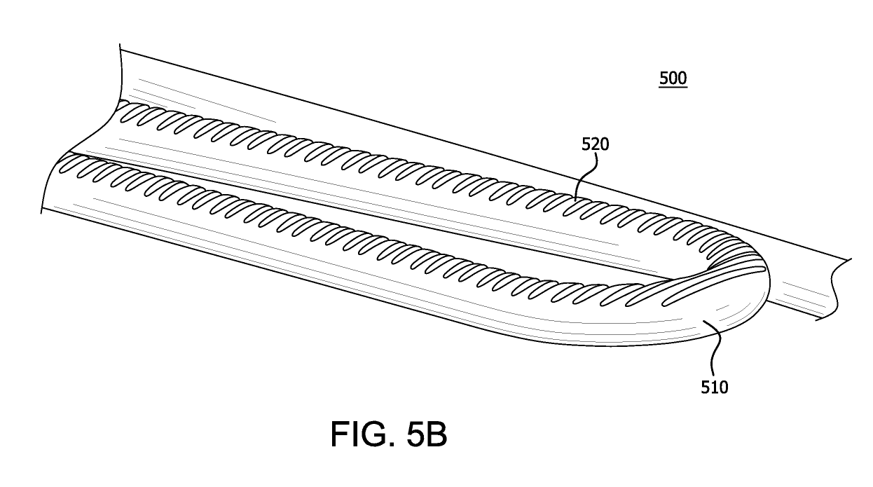

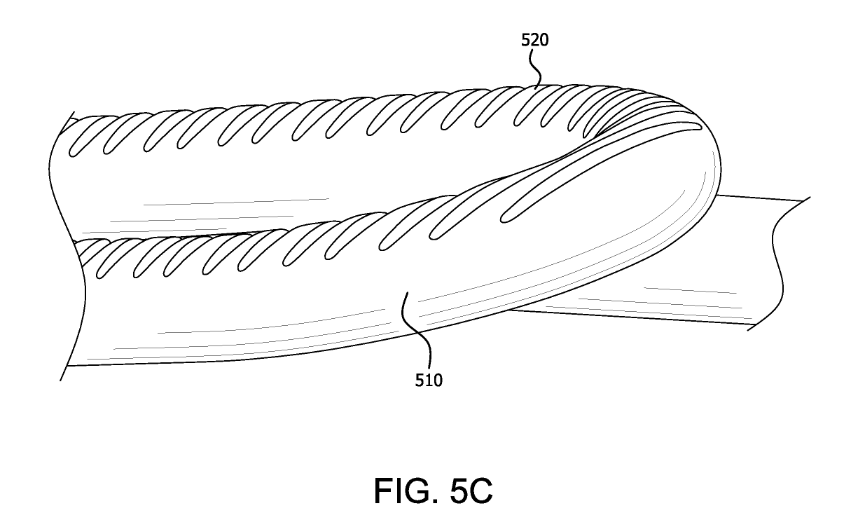

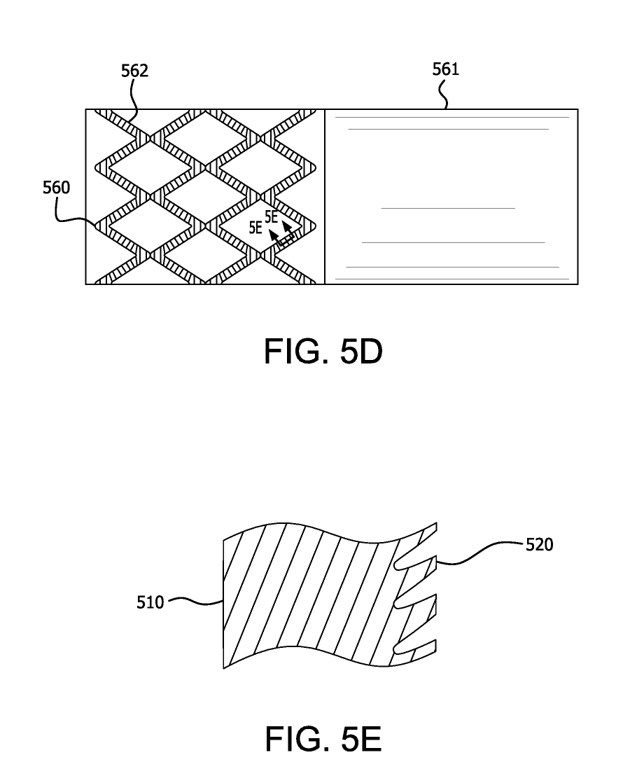

In various embodiments and with reference to FIGS. 5A-5E, a medical device may be provided with a support structure (e.g., frame, stent 562, or the like) or an anchor wire 510. The support structure or anchor wire 510 may be an integral part of the medical device (e.g., a stent 562), and shown in FIG. 5D, or may be an exterior anchoring device. Moreover, the support structure or anchor wire 510 and/or stent sup may comprise one or more tines 520 over a surface of the support structure of anchor wire 510. For example, a medical device 560 (e.g., a stent graft) may comprise a stent 562. Stent 562 may be a wire comprising a plurality of tines 520 and being at least partially covered by a graft material 561, as shown in FIG. 5D and, as shown in greater detail with respect to the plurality of tines 520 in FIG. 5E. Stent 562 may also be configured to engage the anatomy when medical device 560 is implanted. Where stent 562 comprises one or more tines 520, tines 520 may be configured to engage and retain a graft covering 561. Tines 520 may be located on the exterior surface of stent 562 such that graft 561 is engage by tines 520 when graft 561 is coupled to the exterior surface of stent 562. Similarly, tines 520 may be located on the interior surface of stent 562 such that graft 561 is engage by tines 520 when graft 561 is coupled to the interior surface of stent 562. Tines 520 may also engage the anatomy at a treatment site to retain medical device 560 at the treatment site.

Similarly, a medical device may be configured with anchor wire 510. Anchor wire 510 may be wrapped, adhered or otherwise conformed by the exterior surface of the medical device. When implanted, anchor wire 510 may contact the anatomy such that the one or more tines 520 engage the anatomy and retain the medical device in the treatment region where the device is implanted. Moreover, the integral nature of tines 520 in either the support structure of the medical device or an anchoring system provide for a reduced delivery and deployment geometry.

In various embodiments, the anchor system mounted or coupled to a medical device may deploy through any suitable medical device delivery system. The medical device delivery system may comprise one or more catheters, guidewires, or other suitable conduit for delivering the anchor system mounted or coupled to a medical device to a treatment region. In these embodiments, the catheters, guidewires, or conduits may comprise lumens configured to receive inputs and/or materials from the proximal end of the medical device delivery system and conduct the inputs and/or materials to the anchor system mounted or coupled to a medical device at the treatment region.

In various embodiments, various components of the anchor system mounted or coupled to a medical device are steerable. For example, during deployment at a treatment site, one or more of the medical devices may be configured with a removable steering system that allows an end of the medical device to be biased or directed by a user.

For the avoidance of doubt, the anchoring system and various associated medical may provide therapy to the anatomy and it should be understood that these devices may be implantable in any suitable body lumen or region.

The hooks, medical devices, support structures, coatings, and covers, described above, can be biocompatible. As used herein, “biocompatible” means suited for and meeting the purpose and requirements of a medical device, used for either long or short term implants or for non-implantable applications. Long term implants are defined as items implanted for more than 30 days. These support structures, coatings, and secondary structures may be formed of a fluoropolymer such as ePTFE. Alternatively, or in combination with a fluoropolymer, the support structures, coatings, and secondary structures may be formed of biocompatible materials, such as polymers, which may include fillers such as metals, carbon fibers, Dacron, glass fibers or ceramics. Such polymers may include olefin polymers, polyethylene, polypropylene, polyvinyl chloride, polytetrafluoroethylene which is not expanded, fluorinated ethylene propylene 45 copolymer, polyvinyl acetate, polystyrene, poly(ethylene terephthalate), naphthalene dicarboxylate derivatives, such as polyethylene naphthalate, polybutylene naphthalate, polytrimethylene naphthalate and trimethylenediol naphthalate, polyurethane, polyurea, silicone rubbers, polyamides, polycarbonates, polyaldehydes, natural rubbers, polyester copolymers, styrene-butadiene copolymers, polyethers, such as fully or partially halogenated polyethers, copolymers, and combinations thereof. Also, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, and natural silk may be included in support structures, coatings and secondary structures.

These hooks, medical devices, covers and coatings may be utilized with bio-active agents. Bio-active agents can be coated onto a portion or the entirety of the support structures, coatings and secondary structures for controlled release of the agents once the support structures, coatings and secondary structures is implanted. The bio-active agents can include, but are not limited to, vasodilator, anti-coagulants, such as, for example, warfarin and heparin. Other bio-active agents can also include, but are not limited to agents such as, for example, anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

Thus, the hooks and/or anchoring systems described herein provide mechanisms for creating customizable anchoring systems and for reducing the delivery and deployment geometry of implantable medical devices with anchoring systems.

Numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, materials, elements, components, shape, size and arrangement of parts including combinations within the principles of the invention, to the full extent indicated by the broad, general meaning of the terms in which the appended claims are expressed. To the extent that these various modifications do not depart from the spirit and scope of the appended claims, they are intended to be encompassed therein.

What is claimed is:

1. A medical device anchor for anchoring a medical device to tissue, said medical device anchor comprising:
a body coupled to a medical device with at least one of an adhesive and a graft material;
a hinge portion integral to the body;
a tine coupled to the body though the hinge portion;
the body, the hinge portion and the tine being formed from a sheet of thin-film metal.

2. The medical device anchor of claim 1, wherein the body is in a first plane and the tine extends outwardly from the first plane.

3. The medical device anchor of claim 1, wherein the tine is in a second plane, wherein the first plane and the second plane intersect.

4. The medical device anchor of claim 1, wherein the body comprises a bracket.

5. The medical device anchor of claim 4, wherein the bracket defines a first opening and a second opening, and wherein the first opening and the second opening are separated by a retention member.

6. The medical device anchor of claim 5, wherein an attachment material is configured to pass through the first opening in a first direction, engage the retention member, and pass through the second opening in a second direction.

7. The medical device anchor of claim 6, wherein the medical device comprises a graft having a graft material.

8. The medical device anchor of claim 7, wherein the attachment material is the same as the graft material.

9. The medical device anchor of claim 8, wherein the attachment material is part of the graft material.

10. The medical device anchor of claim 8, wherein the graft material is ePTFE.

11. An anchoring system created by a method comprising:
masking at least one of a sheet and a tube of thin-film nitinol to define a tine geometry and a body geometry, wherein a portion of the at least one of the sheet and the tube is not masked;
exposing the at least one of the sheet and the tube to a solvent, wherein the portion of the at least one of the sheet and the tube that is not masked is dissolved by the solvent;
subjecting at least one of a first portion of the at least one of the sheet and the tube associated with the tine geometry and a second portion of the at least one of the sheet and the tube associated with the body geometry to an energy source to bias the first portion out of phase with the second portion.

12. The method of claim 11, wherein the anchoring system is subjected to a heat treatment.

13. The method of claim 12, wherein the heat treatment creates a stress concentration region.

14. The method of claim 13, wherein the stress concentration region defines a predetermined failure mode in response to an over-stress condition.

15. The method of claim 11, further comprising;
providing a medical device; and
attaching the anchoring system to the medical device with at least one of an adhesive and a graft material.

16. The method of claim 11 wherein the energy source is a laser.

17. The method of claim 11 wherein the energy source is a pressurized fluid.

18. A medical device, comprising:
an outer surface comprising a graft material;
a plurality of hooks adhered to the graft material, wherein the plurality of hooks have a hook density of at least 1600 hooks per square mm.

19. A medical device, comprising:
an outer surface comprising a graft material;
a plurality of hooks adhered to the graft material, wherein the plurality of hooks have a hook density of about 2,000 hooks per square mm.

20. A medical device, comprising:
a stent having an exterior surface and a first generally cylindrical shape having a longitudinal centerline;
a graft attached to the exterior surface;
an anchoring system comprising:
a first plurality of hooks biased away from the longitudinal centerline, the first plurality of hooks configured to engage a treatment region of an anatomy in response to being implanted in the anatomy at the treatment region; and
a second plurality of hooks biased toward the longitudinal centerline, the second plurality of hooks configured to engage at least a portion of the graft.

21. The medical device of claim 20, wherein the anchoring system has a second generally cylindrical shape that substantially approximates the first generally cylindrical shape of the stent.

22. The medical device of claim 20, wherein the second plurality of hooks attached the anchoring system to the graft.

23. The medical device of claim 20, wherein the anchoring system is adhered to the graft with an adhesive.

24. The medical device of claim 20, wherein the anchoring system is formed from a tube of thin-film nitinol.

25. The medical device of claim 20, wherein the anchoring system is formed from a shape memory material.

26. An anchoring system created by a method comprising:
cutting at least one of a sheet and a tube of thin-film nitinol to define a tine geometry and a body geometry, wherein a portion of the at least one of the sheet and the tube is not cut;
subjecting at least one of a first portion of the at least one of the sheet and the tube associated with the tine geometry and a second portion of the at least one of the sheet and the tube associated with the body geometry to an energy source to bias the first portion out of phase with the second portion.

27. The method of claim 26 wherein the energy source is a laser.

28. The method of claim 26 wherein the energy source is a pressurized fluid.
